(19) United States
(12) Patent Application Publication
(10) Pub. No.: US 2003/0032935 A1
(43) Pub. Date: Feb. 13, 2003

(54) Packages facilitating convenient mixing and delivery of liquids

(75) Inventors: Louis F. Damiano JR., Westminster, MA (US); James P. Delaney, Billerica, MA (US); John L. Croteau, Woonsocket, RI (US)

Correspondence Address: FISH & RICHARDSON PC, 225 FRANKLIN ST, BOSTON, MA, 02110, US

(73) Assignee: SciMed Life Systems, Inc., Maple Grove, MN (US)

(21) Appl. No.: 10/158,642

(22) Filed: May 30, 2002

Related U.S. Application Data
(60) Provisional application No. 60/311,602, filed on Aug. 10, 2001.

(51) Int. Cl. A61B 19/00

(52) U.S. Cl. 604/403; 604/408; 604/415

(57) ABSTRACT

Embolic devices and methods for mixing and delivering embolic material in a sterile environment facilitate delivery of the embolic material directly into a patient thereby preventing the embolic material from becoming contaminated. Such devices include a sealable container couplable to a syringe, a dissolvable caplet or gel-cap including a solid or liquid embolic material, a sealed vial with a breakable neck containing an embolic material, and a flexible container including internal compartments separated by breakable membranes.

### Cross Reference to Related Case

This application claims priority to and the benefit of Provisional U.S. Patent Application Serial No. 60/311,602, filed Aug. 10, 2001, the entirety of which is hereby incorporated by reference.

### FIELD OF THE TECHNOLOGY

The invention relates generally to embolic devices and more specifically to devices for mixing and delivering embolic material.

### BACKGROUND OF THE TECHNOLOGY

Embolization is the therapeutic introduction of various substances (embolic material) into a patient's circulatory system for the purpose of occluding vessels, either to arrest or to prevent hemorrhaging or to defunctionalize a structure or an organ.

Typical methods of introducing embolic material, such as spherical polyvinyl alcohol for example, require a user to peel or unscrew a cap off of a container and pour the embolic material out of the container into a mixing vessel. The embolic material, after being mixed with a carrier material, such as ethanol, and a hydrating material, such as saline, may then be introduced into a syringe and subsequently injected into a catheter. This procedure is inconvenient and potentially wasteful.

### SUMMARY

The present invention relates to embolic devices for mixing and delivering embolic material. Specifically, the embolic devices facilitate mixing of the embolic material in a sterile environment and delivery of the embolic material directly into a patient, thereby preventing the embolic material from becoming contaminated.

Accordingly, in one aspect, the invention involves a method of mixing and dispensing an embolic material. The method includes providing a container having a luer fitting and containing an embolic material; transferring at least one fluid from a syringe into the container via the luer fitting, and agitating the container to mix the embolic material and the fluid(s). The method further includes transferring at least a portion of the mixed material into a syringe; and administering the mixed material from the syringe.

In another aspect, the invention utilizes a flexible container including a plurality of internal compartments separated by breakable membranes; the container also includes a sealed fluid connection. One of the compartments contains an embolic material and each other compartment contains a material to be mixed therewith. Pressure is applied so as to break at least one of the seals, and the flexible container is kneaded to mix contents from the compartments separated by the at least one broken seal. The method still further includes unsealing the fluid connection and dispensing the mixed contents therethrough for administration.

In one embodiment, the container includes a plurality of breakable membranes and the pressure-applying and kneading steps are repeated in a sequential manner so as to mix the contents of two adjacent compartments before another breakable membrane is broken. Indeed, the contents of all compartments may be combined prior to the kneading step.

In still another aspect, the invention involves providing a caplet including an embolic material surrounded by an inert dissolvable material, dissolving the caplet in a fluid to form a mixture, and administering the mixture. In one embodiment, the caplet is dissolved in a liquid including saline.

In yet another aspect, the invention involves a method of mixing and dispensing an embolic material. The method includes providing a caplet including an embolic material in solid form, dissolving the caplet in a fluid to form a mixture, and administering the mixture. In one embodiment, the caplet is dissolved in a liquid including saline.

In another aspect, the invention involves providing a sealed, blow-molded vial which contains an embolic material. The vial includes a luer connector and, thereover, a breakable fitting providing a fluid seal over the luer connector. The method further includes transferring the embolic material to a syringe via the luer fitting and administering the mixed material from the syringe.

In still another aspect, the invention involves a medical device including a container for holding an embolic material and a cap including a luer fitting. The cap facilitates removable coupling of a syringe barrel to the container to selectively seal and unseal the container.

In yet another aspect, the invention involves a medical device including a flexible outer package. The medical device further includes plurality of membranes disposed within the flexible outer package and positioned to define therein a plurality of compartments. At least one of the plurality of membranes is breakable when pressure is applied to a compartment bounded by the at least one membrane, thereby allowing contents from the compartment to mix with contents of an adjacent compartment. The medical device further includes a sealable outlet for dispensing the mixed contents.

In another aspect, the invention involves a medical device including a dissolvable solid material defining an enclosed cavity, and a solid or liquid embolic material disposed within the enclosed cavity.

In still another aspect, the invention involves a medical device including a sealed vial including an interior, a connector, and a flexible and breakable seal over the connector. When the neck is broken, access is afforded to the interior of the vial via the connector. The medical device further includes an embolic material disposed in the interior of the vial.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the technology.

FIG. 1A is a perspective view of an embolic material mixing and/or delivery device including a cap with a luer fitting and a container coupled to a syringe according to one embodiment of the invention.

FIG. 1B is a perspective view of an embolic material mixing and/or delivery device including a cap with a luer fitting and a container according to another embodiment of the invention.

FIG. 1C is a perspective view of the syringe shown in FIG. 1A directly connected to a catheter disposed in a patient.

FIGS. 2A-2C are plan views of embolic material mixing and delivery devices including a flexible outer package with a plurality of internal compartments and a luer connector according to another embodiment of the invention.

FIG. 2D is a plan view of the embolic material mixing and delivery device shown in FIGS. 2A-2C connected to a catheter disposed in a patient.

FIG. 3 is a perspective view of a capsule package for embolic material according to yet another embodiment of the invention.

FIG. 4A is a perspective view of a squirt vial package for delivering embolic material according to still another embodiment of the invention.

FIG. 4B is a perspective view of a squirt vial package including a luer connector according to yet another embodiment of the invention.

FIG. 4C is a perspective view of the squirt vial package shown in FIG. 4B directly connected to a catheter disposed in a patient.

### DETAILED DESCRIPTION

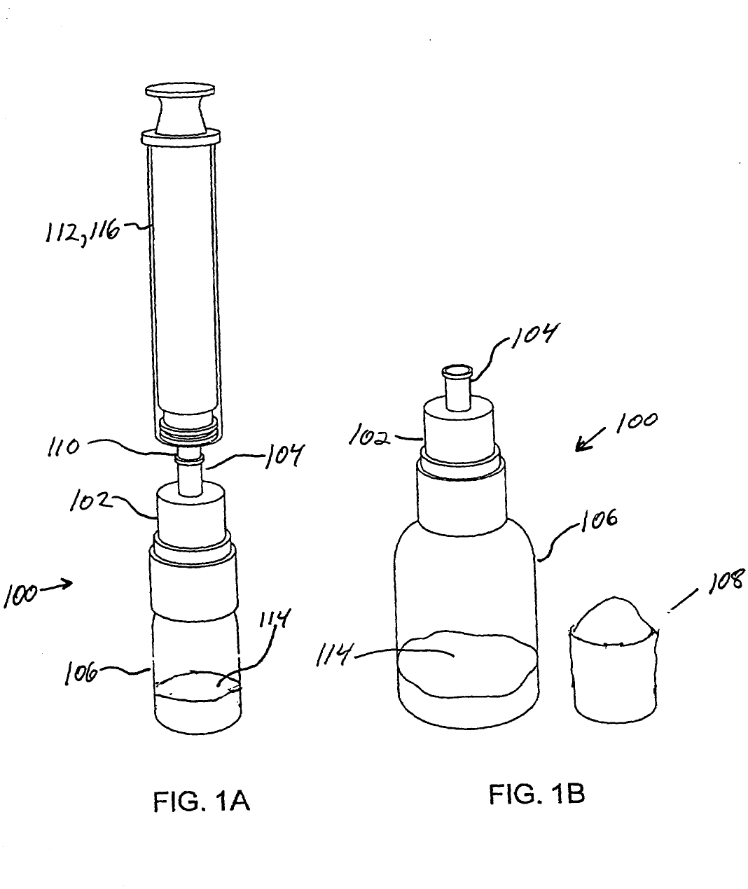

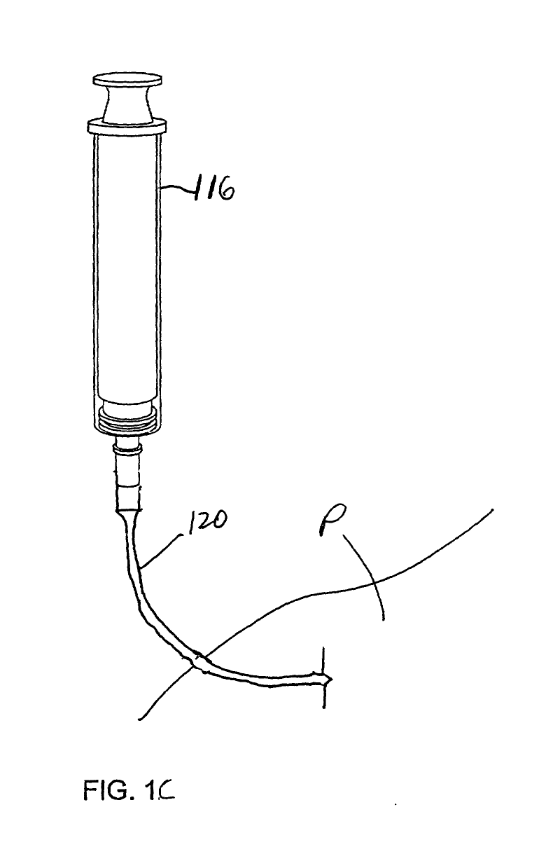

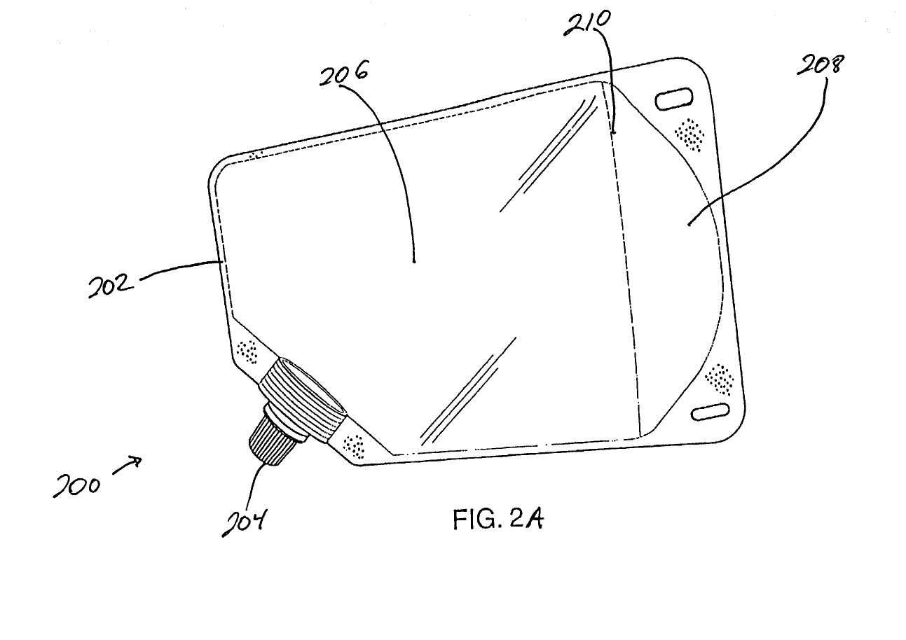

Referring to FIGS. 1A and 1B, in one embodiment, a device 100 in accordance with the invention includes a cap 102 having a luer fitting 104, and a container 106 used for mixing and/or delivering embolic material such as spherical polyvinyl alcohol (S-PVA), for example. In other embodiments, the container 106 can be large or small depending on the amount of embolic material to be mixed. The cap 102 is removably coupled to the container 106 via a threaded connection or a friction connection and seals the container 106. The cap 102 can also include a protective cover 108 which is used to protect the luer fitting 104 when the luer fitting 104 is not in use. The luer fitting 104 is removably coupled to the barrel 110 of a syringe 112 and allows a user to transfer fluid (such as embolic material) from the container 106 to the syringe 112 or vice versa. In various embodiments, the container 106 can be made of glass or a nonleaching and/or nonextracting plastic composition.

In typical operation, the container 106 is partially filled with an embolic material 114. The user attaches the barrel 110 of the syringe 112 to the luer fitting 104 and injects a hydrating liquid and/or other liquid, such as a contrast agent, into the container 106 holding the embolic material. The user may repeat this procedure to add other fluids (e.g. saline) to the container 106. The user then detaches the barrel 110 of the injection syringe 112 from the luer fitting 104 and shakes or agitates the container 106 (e.g., with the cap 108 in place), thereby mixing the embolic material with the newly added liquid(s). The user next attaches to the luer fitting 104 a new syringe 116, turns the container 106 upside down and aspirates the mixture out of the container 106 into the syringe 116.

Referring to FIG. 1C, the user can then attach the syringe 116 to a catheter 120 introduced within a patient P and administer the mixture.

This embodiment of the invention does not require the cumbersome procedures of the prior art, or the use of complex valves (such as a three-way stopcock) to mix the embolic material with other liquids, and provides a convenient and sterile mixing container (i.e., container 106). This embodiment also facilitates easy injection and removal of liquids from the container 106 and provides a closed system for handling the embolic material 114.

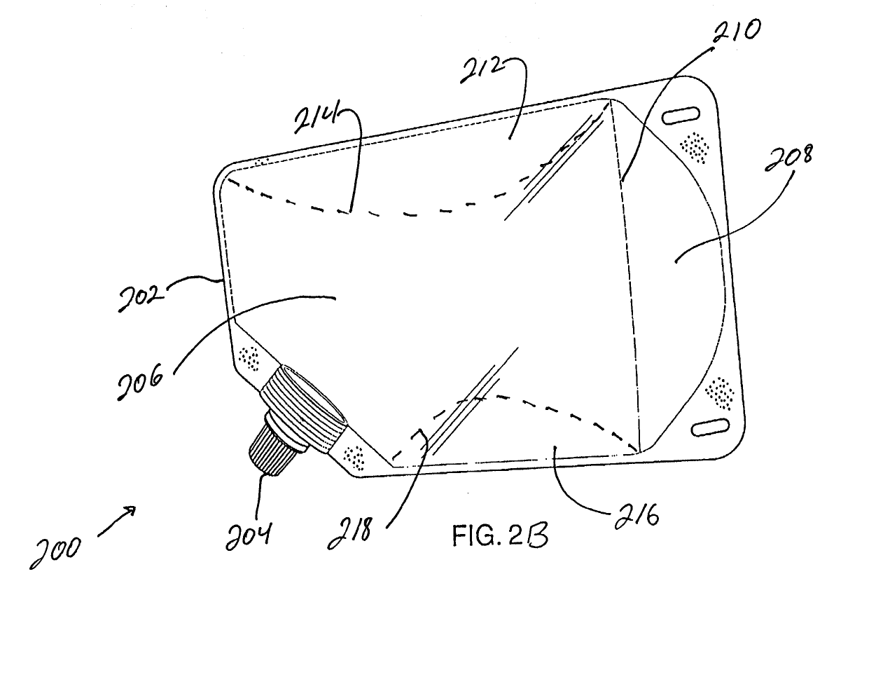

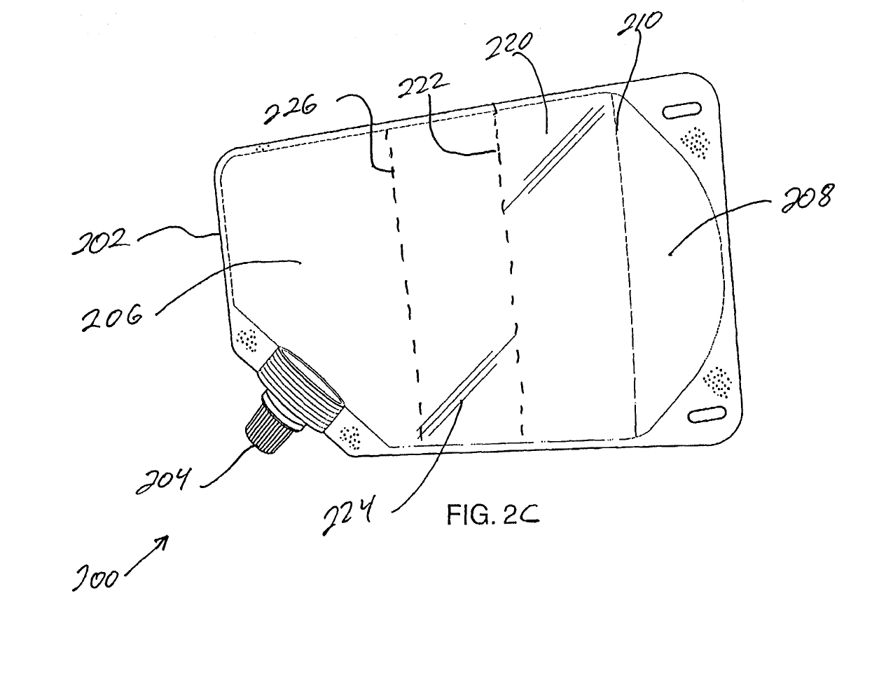

Referring to FIG. 2A, in another embodiment, a device 200 includes a flexible outer container (e.g., plastic, for example) or package 202 with a plurality of internal compartments 206, 208 and a sealable connector 204 in fluid communication with one of the compartments (e.g. a main compartment 206). Each of the internal compartments 206, 208 is separated by a breakable seal 210. In one embodiment, the breakable seal 210 is created by heat sealing the package 202 where the breakable seal 210 is desired. The contents that are to be stored in the compartment 208 are put into the package 202. The breakable seal 210 is then created by heat sealing the package 202 to isolate the contents in the compartment 208 from the compartment 206. The contents that are to be stored in compartment 206 are then put into the package 202. This process may be repeated to create additional compartments. In other embodiments, the breakable seal 210 can be a thin plastic membrane. In some embodiments, the package 202 is made of single or multilayer extrusions of inert polymeric materials.

Referring to FIG. 2B, in another embodiment, a series of internal compartments 208, 212, 216 is positioned around the perimeter of the package 202 and around the central compartment 206. The central compartment 206 is separated from the perimeter compartments 208, 212, 216 by breakable seals 210, 214, 218.

Referring to FIG. 2C, in still another embodiment, the internal compartments 206, 208, 220, 224 is positioned one after the other. This configuration is useful when the contents of the compartments are intended to be mixed in a particular sequence. For example, the contents of compartment 206 and compartment 224 can be mixed together by breaking the seal 226. Likewise, the contents of compartment 208 and compartment 220 can be mixed by breaking the seal 201. The two new mixtures are still separated by seal 222 and can be mixed when desired by breaking the seal 222.

Referring again to FIG. 2B, typically, each of the plurality of internal compartments 206, 208, 212, 216 contains one or more solid or liquid component(s) and all the components are intended to be mixed together. For example, one of the plurality of internal compartments 206, 208, 212, 216 can contain an embolic material such as S-PVA in liquid or solid form. Another of the plurality of internal compartments 206, 208, 212, 216 can contain saline, and still another of the plurality of internal compartments 206, 208, 212, 216 can contain a contrast agent.

In operation, the user, in order to mix the contents of the separate compartments 206, 208, 212, 216, squeezes a particular compartment (e.g. 208, for example), and thereby breaks the seal 210 between the squeezed compartment and an adjacent compartment 206. The user then kneads the flexible outer package 202 until the components from the adjacent compartments 206, 208 are mixed. For example, the user can squeeze the compartment 208 containing the embolic material so as to break the seal 210 between the compartment 208 containing the embolic material and the compartment 206 containing the saline. After kneading the flexible outer package 202 until the embolic material and the saline are properly mixed, the user can squeeze the compartment 212 containing the contrast agent so as to break the seal 214. The user again kneads the flexible outer package 202 until the contrast agent, the embolic material, and the saline are properly mixed. Thereafter, the user can unseal the connector 204 (i.e. remove or unscrew a cap) and dispense the mixture into a desired container for delivery into a patient.

Referring to FIG. 2D, the sealable connector 204 can be a luer fitting that is capable of being attached directly to a catheter 230 (or syringe, or other medical device with a luer fitting) that is introduced in a patient P.

Advantages of the flexible package 202 with the plurality of compartments 206, 208, 212 include having all the separate components of an embolic mixture conveniently contained within one package, which itself facilitates mixing of the components without exposing any of them to an external environment or requiring the use of additional mixing containers.

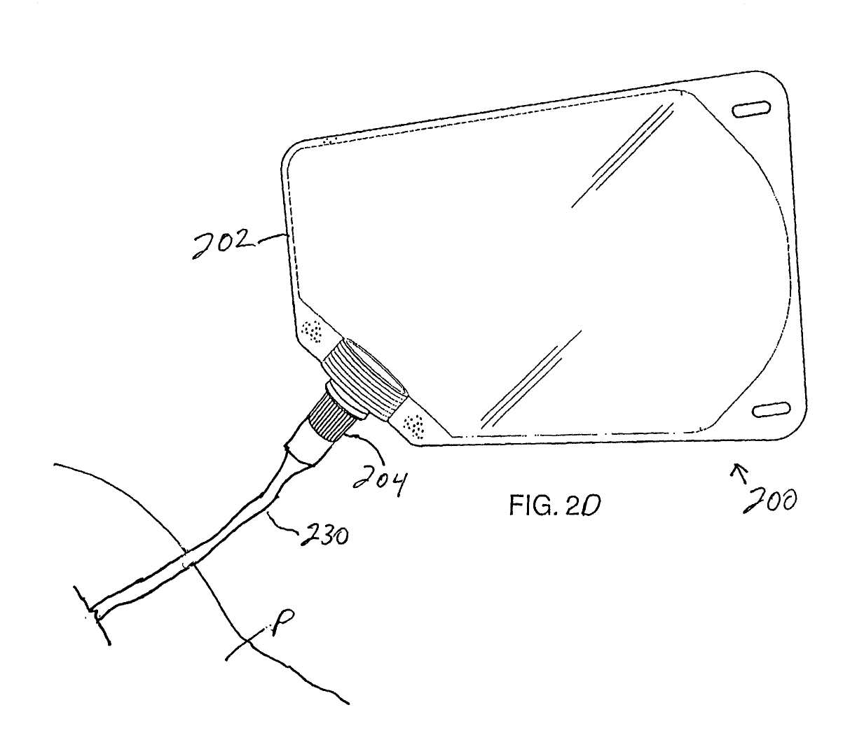

Referring to FIG. 3, in still another embodiment of the invention, a device 300 includes either liquid or solid embolic material 304 (such as S-PVA) encapsulated in a dissolvable solid material (e.g., a gel-cap) 302 or a solid embolic material formed into a caplet 306. The gel-cap 302 or caplet 306 can be dissolved in a saline solution. After the gel-cap 302 or caplet 306 has been dissolved, the resulting embolic mixture can be drawn into a syringe or poured into some other delivery device and then introduced into a patient through a catheter. Additionally, the gel-cap 302 material is made of an inert substance that does not affect the embolic mixture upon dissolving. The gel-caps 302 or caplets 306 are stored in protective package 308 until needed. 5 In one embodiment, the protective package 308 is a plastic bubble package 310 with a paper or foil backing 312. A gel-cap 302 or caplet 306 is removed from the plastic bubble package 310 by pressing or crushing a particular bubble, thereby forcing the gel-cap 302 or caplet 306 through the paper or foil backing 312. Advantages of the dissolvable gel-cap 302 or caplet 306 include easy and efficient storage of the embolic material.

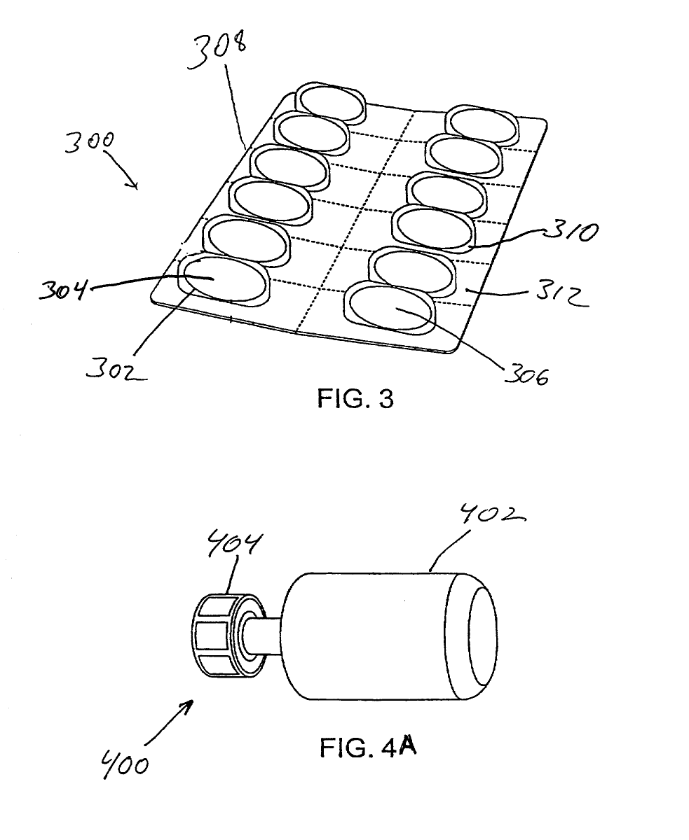

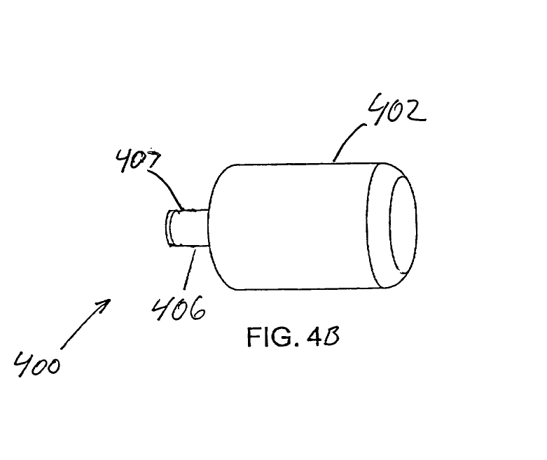

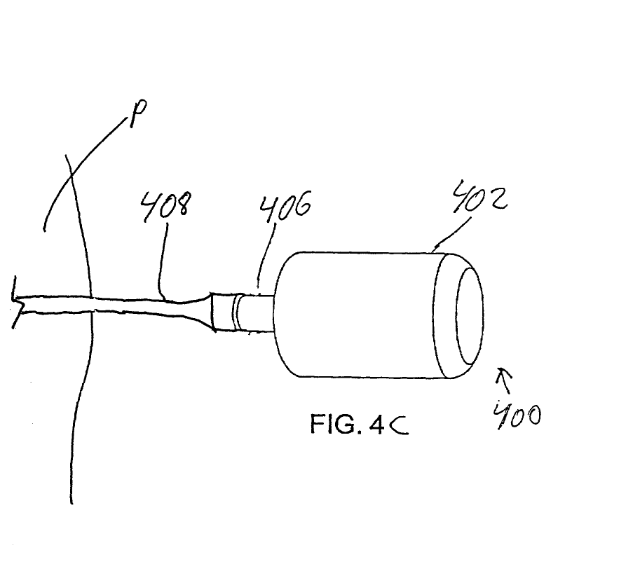

Referring to FIGS. 4A and 4B, in yet another embodiment, a device 400 includes a unit dose bottle 402. The unit dose bottle 402 is a sealed, blow-molded vial that is filed with an embolic material, such as S-PVA, and saline. In operation, the user twists off the flexible neck 404 (or cap) and dispenses the contents of the unit dose bottle into a desired container for delivery into a patient. In various embodiments, the unit dose bottle 402 can be made of polypropylene, polyethylene, or other coextruded materials.

Referring to FIGS. 4B and 4C, in another embodiment, the unit dose bottle 402 includes a breakable neck 407. In operation, the user twists off (or breaks) the neck 408 thereby exposing a luer connector 406 which can be attached directly to a catheter 408 (or syringe, or other medical device with a luer fitting) that is introduced in a patient 410.

Variations, modifications, and other implementations of what is described herein will occur to those of ordinary skill in the art without departing from the spirit and the scope of the technology. Accordingly, the technology is not to be defined solely by the preceding illustrative description

What is claimed is:

1. A method of mixing and dispensing an embolic material, the method comprising the steps of:
a. providing a container having a luer fitting and containing an embolic material;
b. transferring at least one fluid from a syringe into the container via the luer fitting;
c. agitating the container to mix the embolic material and the at least one fluid; and
d. transferring at least a portion of the mixed material into a syringe; and
e. administering the mixed material from the syringe.

2. A method of mixing and dispensing an embolic material, the method comprising the steps of:
a. providing a flexible container comprising (i) a plurality of internal compartments separated by breakable membranes and (ii) a sealed fluid connection, one of the compartments containing an embolic material and each other compartment containing a material to be mixed therewith;
b. applying pressure so as to break at least one of the seals;
c. kneading the flexible container to mix contents from the compartments separated by the at least one broken seal; and
d. unsealing the fluid connection and dispensing the mixed contents therethrough for administration.

3. The method of claim 2 wherein the container comprises a plurality of breakable membranes, the pressure-applying and kneading steps being repeated in a sequential manner so as to mix the contents of two adjacent compartments before another breakable membrane is broken.

4. The method of claim 3 wherein the container comprises a plurality of breakable membranes, the pressure-applying step being repeated so as to combine the contents of all compartments prior to the kneading step.

5. A method of mixing and dispensing an embolic material, the method comprising the steps of:
a. providing a caplet comprising an embolic material surrounded by an inert dissolvable material;
b. dissolving the caplet in a fluid to form a mixture; and
c. administering the mixture.

6. The method of claim 5 wherein the caplet is dissolved in a liquid comprising saline.

7. A method of mixing and dispensing an embolic material, the method comprising the steps of:
a. providing a caplet comprising an embolic material in solid form;
b. dissolving the caplet in a fluid to form a mixture; and
c. administering the mixture.

8. The method of claim 7 wherein the caplet is dissolved in a liquid comprising saline.

9. A method of mixing and dispensing an embolic material, the method comprising the steps of:
a. providing a sealed vial comprising a luer connector and, thereover, a breakable fitting providing a fluid seal over the luer connector, the vial containing an embolic material;
b. transferring the embolic material to a syringe via the luer fitting; and
c. administering the mixed material from the syringe.

10. A medical device comprising:
a. a container for holding an embolic material; and
b. a cap comprising a luer fitting, the cap facilitating removable coupling of a syringe barrel to the container to selectively seal and unseal the container.

11. A medical device comprising:
a. a flexible outer package;
b. a plurality of membranes disposed within the flexible outer package and positioned to define therein a plurality of compartments, at least one of the plurality of membranes being breakable when pressure is applied to a compartment bounded by the at least one membrane, thereby allowing contents from the compartment to mix with contents of an adjacent compartment; and
c. a sealable outlet for dispensing the mixed contents.

12. A medical device comprising:
a. a dissolvable solid material defining an enclosed cavity; and
b. one of a solid and liquid embolic material disposed within the enclosed cavity.

13. A medical device comprising:
a. a sealed vial comprising an interior, a connector and a flexible and breakable seal over the connector, breaking of the neck affording access to the interior of the vial via the connector; and
b. an embolic material disposed in the interior of the vial
